(19) United States
(12) Patent Application Publication
(10) Pub. No.: US 2021/0052892 A1
(43) Pub. Date: Feb. 25, 2021

(54) METHOD FOR TREATING OVERACTIVE BLADDER AND SECONDARY FECAL INCONTINENCE BY NEUROSTIMULATING TWO NERVE SITES

(71) Applicant: Norbert Kaula, Arvada, CO (US)

(72) Inventor: Norbert Kaula, Arvada, CO (US)

(21) Appl. No.: 17/001,582

(22) Filed: Aug. 24, 2020

Related U.S. Application Data
(60) Provisional application No. 62/890,575, filed on Aug. 22, 2019.

(51) Int. Cl. A61N 1/36; A61N 1/05

(52) U.S. Cl. CPC A61N 1/36007; A61N 1/36062; A61N 1/0551

(57) ABSTRACT

The present invention provides a method for treating overactive bladder (OAB) and secondary fecal incontinence. The method includes (i) providing a neurostimulator or a neurostimulating system with two neurostimulation leads; (ii) delivering the two neurostimulation leads to two nerve sites respectively; and (iii) neurostimulating the two nerve sites in a coordinated manner with the two neurostimulation leads.

### CROSS-REFERENCE TO RELATED APPLICATIONS

This application expressly claims the benefits and priority based on U.S. Provisional Application No. 62/890,575 filed Aug. 22, 2019, which is incorporated herein by references as if set forth in its entirety.

### FIELD OF THE INVENTION

The present invention generally relates to a method for treating overactive bladder (OAB) and secondary fecal incontinence by neurostimulating two nerve sites. Some embodiments provide a method for coordinated stimulation of two or more related neural sites, and a device and a system designed for executing the method. Although the invention will be illustrated, explained and exemplified with a combined sacral and epidural cones medullaris neurostimulation for overactive bladder (OAB) Treatment, it should be appreciated that the present invention can also be applied otherwise and/or in other fields.

### BACKGROUND OF THE INVENTION

Overactive bladder (OAB) refers to a group of urinary symptoms. The most common symptoms are frequent and a sudden, uncontrolled need or urge to urinate. Many patients will leak urine when they feel this urge. Another symptom is the need to pass urine many times during the day and night. OAB may be caused by weak muscles, nerve damage, use of medications, alcohol or caffeine, infection, and being overweight.

Treatments such as pelvic floor muscle exercises, medications, and nerve stimulation can reduce or eliminate OAB symptoms. For example, OAB treatments may include behavioral therapies & physical therapy; drugs such as anitcholinergic+β-agonist; intravesical botox, neuromodulation, sacral, and surgery such as augmentation cystoplasty and urinary diversion. Sacral neuromodulation has been shown to be successful using single lead and single nerve site.

Advantageously, the present invention provides a method for treating overactive bladder (OAB) and secondary fecal incontinence by neurostimulating two nerve sites, which exhibits numerous technical merits as compared to the current neurostimulation using a single lead on a single sacral nerve site.

### SUMMARY OF THE INVENTION

One aspect of the present invention provides a method for treating overactive bladder (OAB) and secondary fecal incontinence. The method includes:

(i) providing a neurostimulator or a neurostimulating system with two neurostimulation leads;

(ii) delivering the two neurostimulation leads to two nerve sites respectively; and

(iii) neurostimulating the two nerve sites in a coordinated manner with the two neurostimulation leads.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

The present invention is illustrated by way of example, and not by way of limitation, in the figures of the accompanying, drawings and in which like reference numerals refer to similar elements. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1 is the flow chart of a method for treating overactive bladder (OAB) and secondary fecal incontinence in accordance with an exemplary embodiment of the present invention.

FIG. 2 depicts an implantable pulse generator (IPG) with an epidural lead stimulating at conus medullaris level and a sacral lead stimulating at S3 level in accordance with an exemplary embodiment of the present invention.

FIG. 3 illustrates the neurological relationship between Interomediolateral Neurons Onuf's Nucleus; Dorsolateral Pons; T11-L2 Spinal Cord Conus Medullaris; Bladder; Urethra; DRG; and S2-S4 Spinal Cord in accordance with an exemplary embodiment of the present invention.

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement.

Where a numerical range is disclosed herein, unless otherwise specified, such range is continuous, inclusive of both the minimum and maximum values of the range as well as every value between such minimum and maximum values. Still further, where a range refers to integers, only the integers from the minimum value to and including the maximum value of such range are included. In addition, where multiple ranges are provided to describe a feature or characteristic, such ranges can be combined.

It is also to be understood that the terminology used herein is for the purpose of describing particular embodiments only, and is not intended to limit the scope of the invention. For example, when an element is referred to as being “on”, “connected to”, or “coupled to” another element, it can be directly on, connected or coupled to the other element or intervening elements may be present. In contrast, when an element is referred to as being “directly on”, “directly connected to”, or “directly coupled to” another element, there are no intervening elements present.

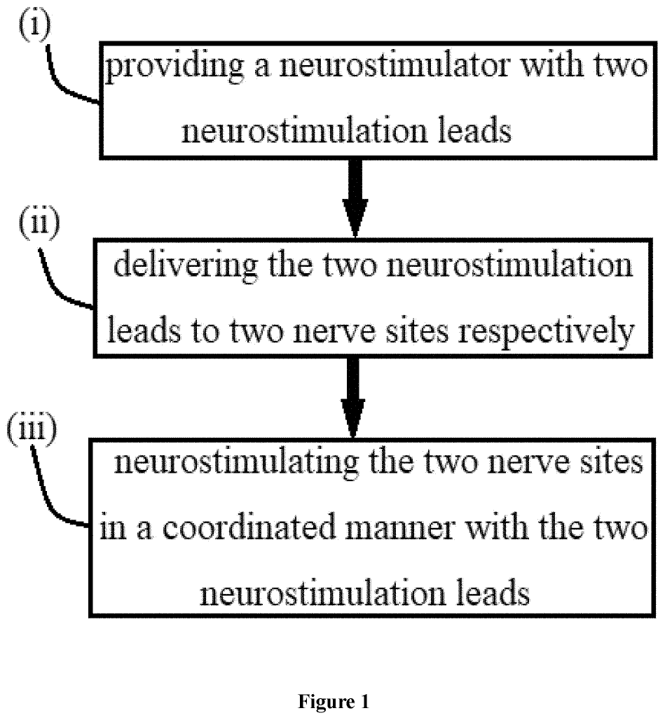

Referring to FIG. 1, various embodiments of the invention provide a method for treating overactive bladder (OAB) and secondary fecal incontinence. The method includes:

(i) providing a neurostimulator or a neurostimulating system with two neurostimulation leads;

(ii) delivering the two neurostimulation leads to two nerve sites respectively; and

(iii) neurostimulating the two nerve sites in a coordinated manner with the two neurostimulation leads.

Traditional techniques in transforaminal sacral spinal nerve stimulation may be employed in the present invention, although they may be described in simplified form, omitted, or merely suggested, in order to avoid unnecessarily obscuring the present invention.

The neurostimulating in step (iii) may be for example sacral and epidural conus medullaris neurostimulation. The two nerve sites are the sacral spinal nerve and the interomediolateral neurons and the Onuf's nucleus, which reside at the conus medullaris (T11-L2) of the spinal cord and can be reached via the epidural space, similar to an epidural pain block. Two percutaneous leads may be inserted into the epidural space to reach these neural fibers.

The invention provides combined sacral and epidural conus medullaris neurostimulation for overactive bladder (OAB) treatment. Stimulating the sacral nerve does not address interomediolateral Neurons and Onuf's nucleus, that reside at the conus medullaris (T11-L2) of the spinal cord.

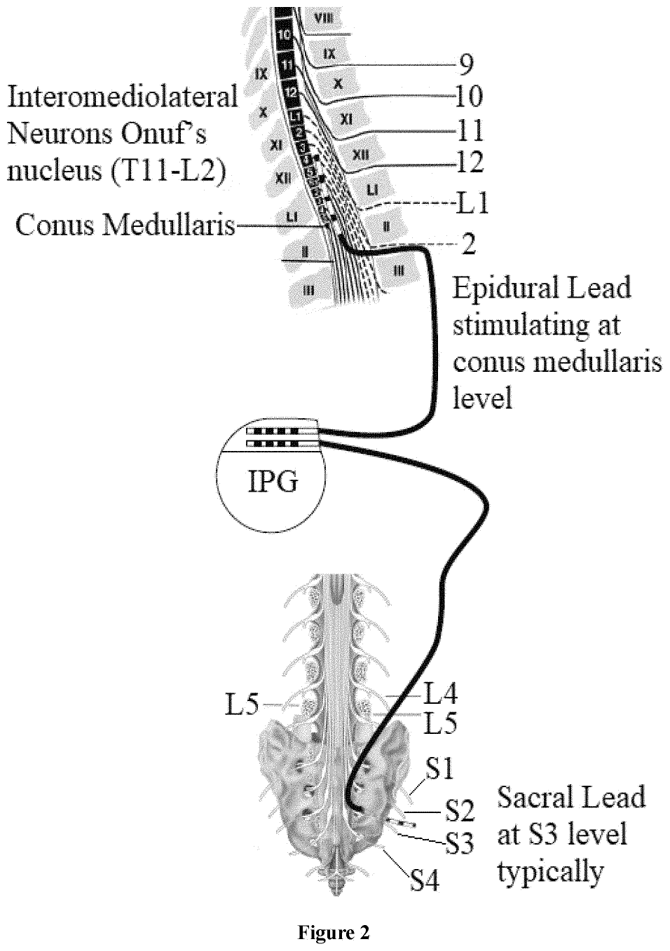

FIG. 2 shows an electronic system such as an implantable pulse generator (IPG) and two implantable leads, one of which is an epidural lead stimulating at corms medullaris level, and another one of which is a sacral lead stimulating at S3 level typically. FIG. 2 also indicates Interomediolateral Neurons Onuf's nucleus (T11-L2) and Conus Medullaris.

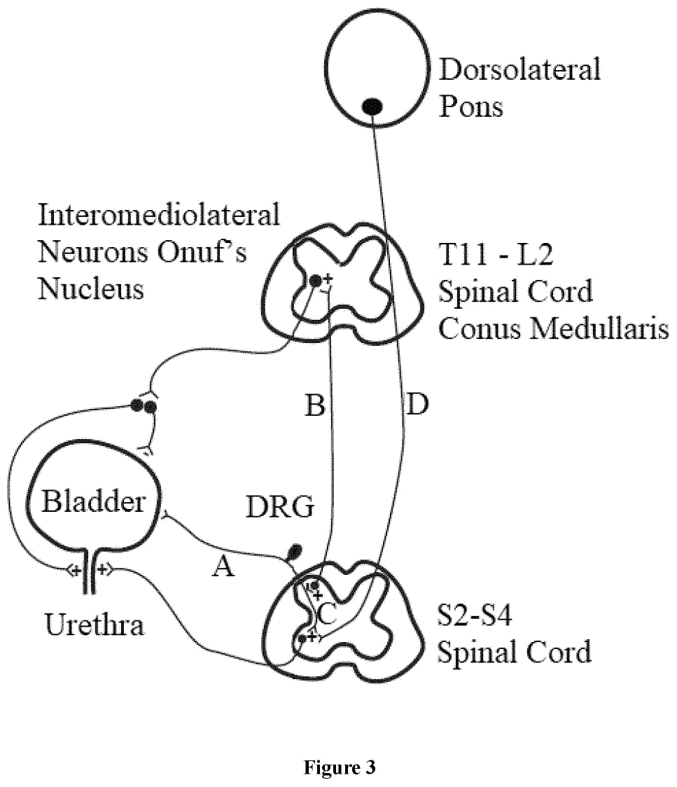

FIG. 3 shows the neurological relationship between Interomediolateral Neurons Onuf's Nucleus; Dorsolateral Pons; T11-L2 Spinal Cord Conus Medullaris; Bladder; Urethra; DRG; and S2-S4 Spinal Cord. As shown in FIG. 3, neural control of the lower urinary tract may include (A) bladder storage reflexes entering the dorsal horn of S2-S4, (B) the interneurons are intraspinally connected to T11-L2 (conus medullaris) to sympathetic neurons of interomediolateral cells; and (C) Descending fibers from the dorsolateral pans can also excite Onuf's nucleus. With increased sympathetic activity, detrusor contraction is inhibited and urethral resistance is increased to prevent urine loss.

The neurostimulating in step (iii) may include, but are not limited to: (1) Sacral+Conus Medullaris simultaneously; (2) Sacral+Conus Medullaris alternating, (3) stimulation sacral—sensing evoked AP Conus M: • use latency time differences for nerve fiber selection, • use evoked AP for Epidural lead placement guidance, and • use sensing to create inhibitory stimulation waveform; and (4) stimulation Conus M—sensing evoked AP sacrum.

In the foregoing specification, embodiments of the present invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicant to be the scope of the invention, is the literal and equivalent scope of the set of claims that issue from this application, in the specific form in which such claims issue, including any subsequent correction.

1. A method for treating overactive bladder (CAB) and secondary fecal incontinence, comprising:
(i) providing a neurostimulator or a neurostimulating system with two neurostimulation leads;
(ii) delivering the two neurostimulation leads to two nerve sites respectively; and
(iii) neurostimulating the two nerve sites in a coordinated manner with the two neurostimulation leads.

2. The method according to claim 1, wherein said neurostimulating is sacral and epidural conus medullaris neurostimulation.

3. The method according to claim 1, wherein the two sites are the sacral spinal nerve and the interomediolateral neurons and the Ones nucleus.

4. The method according, to claim 3, wherein the two sites reside at the conus medullaris (T11-L2) of the spinal cord and can be reached via the epidural space, similar to an epidural pain block.

5. The method according, to claim 1, wherein the two neurostimulation leads are the two implantable leads, one of which is an epidural lead stimulating at conus medullaris level, and another one of which is a sacral lead stimulating at S3 level.
